(19) United States
(12) Patent Application Publication
(10) Pub. No.: US 2022/0125619 A1
(43) Pub. Date: Apr. 28, 2022

(54) MALE CATHETER WITH UNDERGARMENT

(71) Applicant: BENJAMIN RAMIREZ-FEBRES, Bronx, NY (US)

(72) Inventor: BENJAMIN RAMIREZ-FEBRES, Bronx, NY (US)

(21) Appl. No.: 17/080,919

(22) Filed: Oct. 27, 2020

(51) Int. Cl. A61F 5/453; A61F 5/44; A61F 5/443

(52) U.S. Cl. CPC A61F 5/453; A61F 5/443; A61F 5/4408

(57) ABSTRACT

The male catheter with undergarment is a medical device. The male catheter with undergarment is an apparel item. The male catheter with undergarment is worn as underclothing. The male catheter with undergarment is worn over the pelvis of a male patient. The male catheter with undergarment comprises a catheter structure, a garment, and a plurality of quick release buckles. The plurality of quick release buckles attaches the catheter structure to the garment. The catheter structure is a device that captures urine eliminated by the patient. The catheter structure stores the urine eliminated by the patient for subsequent disposal.

### CROSS REFERENCES TO RELATED APPLICATIONS

Not Applicable

### STATEMENT REGARDING FEDERALLY SPONSORED RESEARCH

Not Applicable

### REFERENCE TO APPENDIX

Not Applicable

### BACKGROUND OF THE INVENTION

### Field of the Invention

The present invention relates to the field of medical and veterinary science, more specifically, a genital device adapted for use in receiving collecting urine. (A61F5/451)

### SUMMARY OF INVENTION

The male catheter with undergarment is a medical device. The male catheter with undergarment is an apparel item. The male catheter with undergarment is worn as underclothing. The male catheter with undergarment is worn over the pelvis of a male patient. The male catheter with undergarment comprises a catheter structure, a garment, and a plurality of quick release buckles. The plurality of quick release buckles attaches the catheter structure to the garment. The catheter structure is a device that captures urine eliminated by the patient. The catheter structure stores the urine eliminated by the patient for subsequent disposal. These together with additional objects, features and advantages of the male catheter with undergarment will be readily apparent to those of ordinary skill in the art upon reading the following detailed description of the presently preferred, but nonetheless illustrative, embodiments when taken in conjunction with the accompanying drawings.

In this respect, before explaining the current embodiments of the male catheter with undergarment in detail, it is to be understood that the male catheter with undergarment is not limited in its applications to the details of construction and arrangements of the components set forth in the following description or illustration. Those skilled in the art will appreciate that the concept of this disclosure may be readily utilized as a basis for the design of other structures, methods, and systems for carrying out the several purposes of the male catheter with undergarment.

It is therefore important that the claims be regarded as including such equivalent construction insofar as they do not depart from the spirit and scope of the male catheter with undergarment. It is also to be understood that the phraseology and terminology employed herein are for purposes of description and should not be regarded as limiting.

### BRIEF DESCRIPTION OF DRAWINGS

The accompanying drawings, which are included to provide a further understanding of the invention are incorporated in and constitute a part of this specification, illustrate an embodiment of the invention and together with the description serve to explain the principles of the invention. They are meant to be exemplary illustrations provided to enable persons skilled in the art to practice the disclosure and are not intended to limit the scope of the appended claims.

FIG. 1 is a perspective view of an embodiment of the disclosure.

FIG. 2 is a front view of an embodiment of the disclosure.

FIG. 3 is a side view of an embodiment of the disclosure.

FIG. 4 is an in-use view of an embodiment of the disclosure.

FIG. 5 is a detail view of an embodiment of the disclosure.

FIG. 6 is a detail view of an embodiment of the disclosure.

### DETAILED DESCRIPTION OF THE EMBODIMENT

The following detailed description is merely exemplary in nature and is not intended to limit the described embodiments of the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to practice the disclosure and are not intended to limit the scope of the appended claims. Furthermore, there is no intention to be bound by any expressed or implied theory presented in the preceding technical field, background, brief summary or the following detailed description.

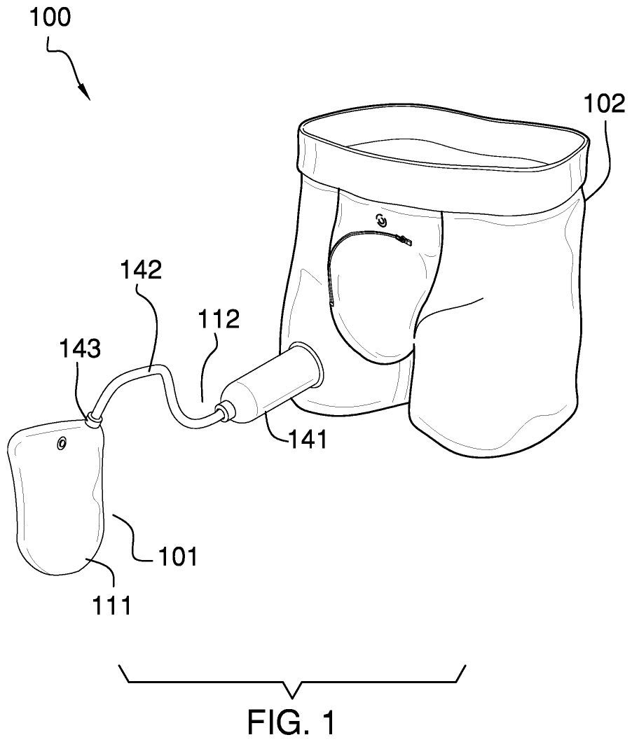

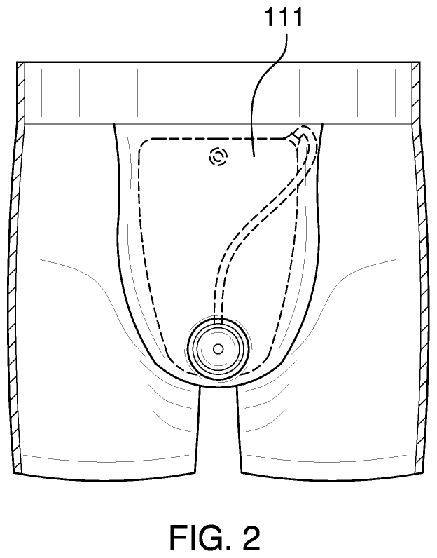

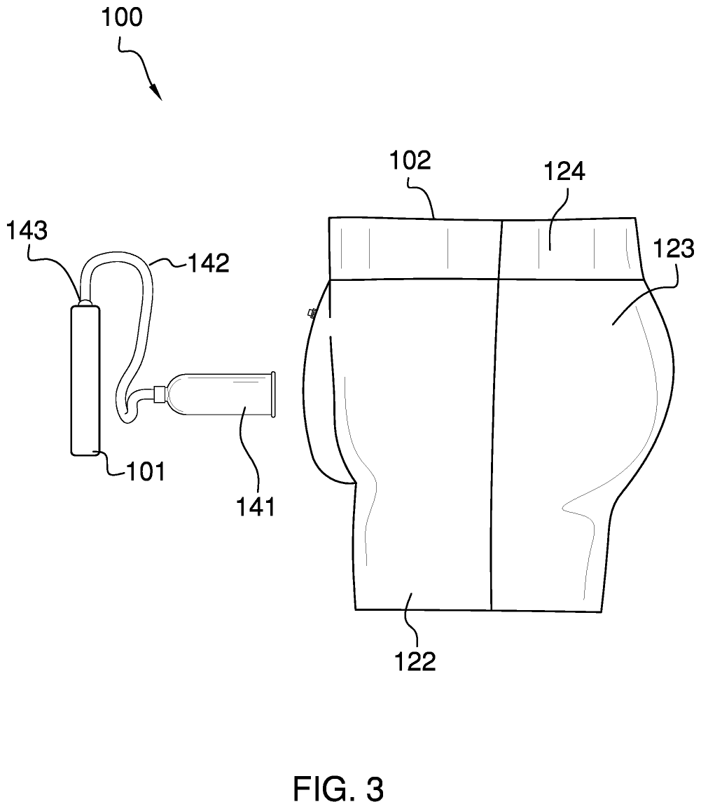

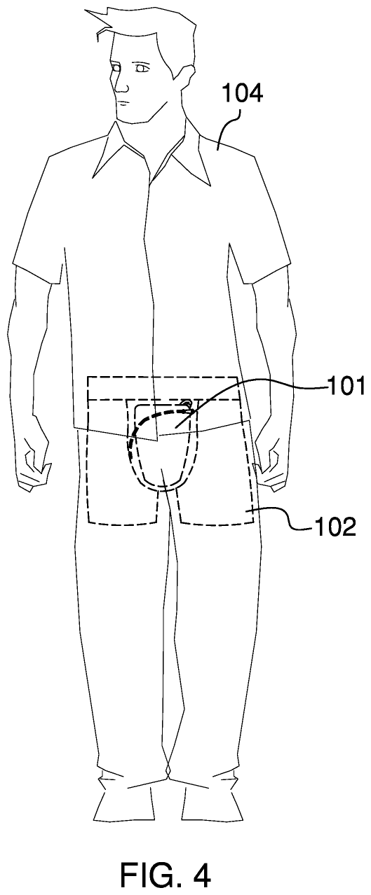

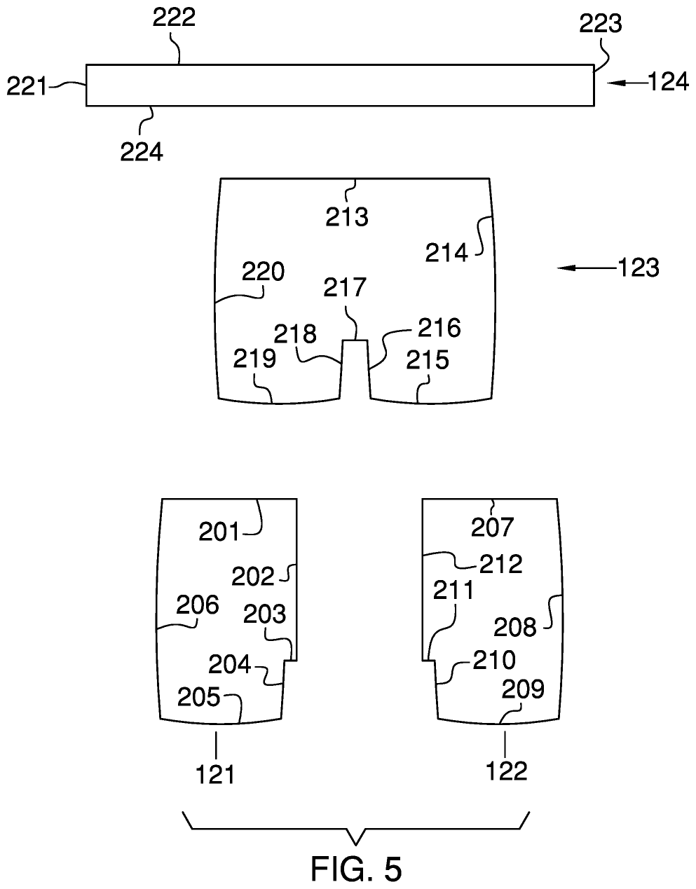

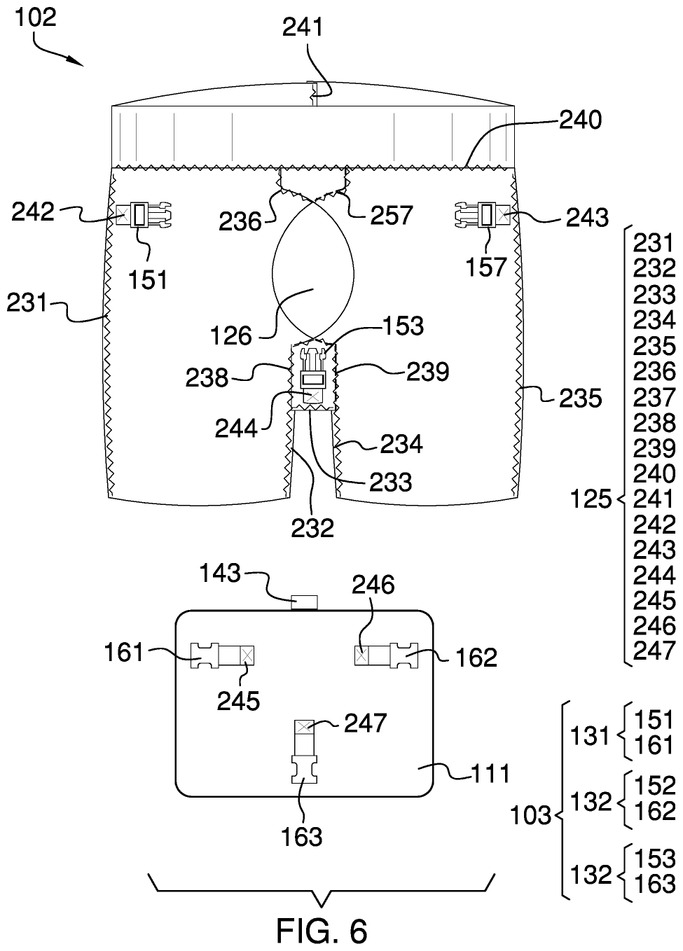

Detailed reference will now be made to one or more potential embodiments of the disclosure, which are illustrated in FIGS. 1 through 6.

The male catheter with undergarment 100 (hereinafter invention) is a medical device. The invention 100 is an apparel item. The invention 100 is worn as underclothing. The invention 100 is worn over the pelvis of a male patient 104. The patient 104 is defined elsewhere in this disclosure. The invention 100 comprises a catheter structure 101, a garment 102, and a plurality of quick release buckles 103. The plurality of quick release buckles 103 attaches the catheter structure 101 to the garment 102. The catheter structure 101 is a device that captures urine eliminated by the patient 104. The catheter structure 101 stores the urine eliminated by the patient 104 for subsequent disposal.

The catheter structure 101 is a medical collection device. The catheter structure 101 is adapted to collect and store excretions from the male patient 104. The catheter structure 101 attaches to the patient 104. The plurality of quick release buckles 103 attaches to the catheter structure 101 to the garment 102. The catheter structure 101 receives excretions released from the urethra of the patient 104. The catheter structure 101 comprises a codpiece 111 and a condom catheter 112.

The codpiece 111 is a bladder. The bladder is defined elsewhere in this disclosure. The codpiece 111 is adapted for use in collecting and storing urine excreted by the patient 104. The plurality of quick release buckles 103 attach the codpiece 111 to the interior panels of the garment 102. The codpiece 111 covers the aperture 126 of the garment 102.

The condom catheter 112 is a medical collection device. The condom catheter 112 forms a fluidic collection between the urethra of the patient 104 and the codpiece 111. The penis of the patient 104 inserts into the condom catheter 112 such that excretions of urine from the urethra of the patient 104 are received directly within the condom catheter 112. The condom catheter 112 transports the received excretions to the codpiece 111. The condom catheter 112 comprises a receiving structure 141, a tube 142, and a fitting 143.

The receiving structure 141 is a tubular structure. The receiving structure 141 is sized such that the penis of the patient 104 inserts into the receiving structure 141. The receiving structure 141 receives the excretions released from the urethra and transports the excretions to the tube 142. The tube 142 is a hose. The tube 142 forms a fluidic connection between the receiving structure 141 and the fitting 143. The tube 142 transports the received excretions to the fitting 143. The fitting 143 is a mechanical structure that attaches the tube 142 to the codpiece 111. The fitting 143 forms a fluidic connection between the tube 142 and the interior of the bladder structure of the codpiece 111 such that the excretions transported by the tube 142 are released into the codpiece 111.

The garment 102 is an item of underclothing. The garment 102 is a textile-based structure. The garment 102 is worn over the pelvis of the patient 104. The garment 102 secures the catheter structure 101 to the patient 104. The garment 102 comprises an interior right panel 121, an interior left panel 122, a posterior panel 123, an elastic webbing 124, and a plurality of seams 125. The garment 102 further forms an aperture 126.

The interior right panel 121 is a textile-based sheeting. The interior right panel 121 is formed in a rectilinear shape. The interior right panel 121 is the portion of the garment 102 that encloses the right interior region of the pelvis of the patient 104. The interior right panel 121 comprises a first edge 201, a second edge 202, a third edge 203, a fourth edge 204, a fifth edge 205, and a sixth edge 206.

The first edge 201, the third edge 203, and the fifth edge 205 of the interior right panel 121 are parallel edges. The second edge 202, the fourth edge 204, and the sixth edge 206 of the interior right panel 121 are parallel edges. The first edge 201 is the edge of the interior right panel 121 selected from the group consisting of the first edge 201, the third edge 203, and the fourth edge 204 with the greatest span of length. The sixth edge 206 is the edge of the interior right panel 121 selected from the group consisting of the second edge 202, the fourth edge 204, and the sixth edge 206 with the greatest span of length.

The fifth edge 205 is the edge of the interior right panel 121 that is distal from the first edge 201. The second edge 202 is the edge of the interior right panel 121 that is distal from the sixth edge 206. The position of the third edge 203 is between the first edge 201 and the fifth edge 205. The fourth edge 204 is between the sixth edge 206 and the second edge 202.

The vertex formed by the first edge 201 and the second edge 202 forms a right angle. The vertex formed by the second edge 202 and the third edge 203 forms a right angle. The vertex formed by the third edge 203 and the fourth edge 204 forms a right angle. The vertex formed by the fourth edge 204 and the fifth edge 205 forms a right angle. The vertex formed by the fifth edge 205 and the sixth edge 206 forms a right angle. The vertex formed by the sixth edge 206 and the first edge 201 forms a right angle.

The interior left panel 122 is a textile-based sheeting. The interior left panel 122 is formed in a rectilinear shape. The interior left panel 122 is the portion of the garment 102 that encloses the left interior region of the pelvis of the patient 104. The interior left panel 122 comprises a seventh edge 207, an eighth edge 208, a ninth edge 209, a tenth edge 210, an eleventh edge 211, and a twelfth edge 212.

The seventh edge 207, the ninth edge 209, and the eleventh edge 211 of the interior left panel 122 are parallel edges. The eighth edge 208, the tenth edge 210, and the twelfth edge 212 of the interior left panel 122 are parallel edges. The seventh edge 207 is the edge of the interior left panel 122 selected from the group consisting of the seventh edge 207, the ninth edge 209, and the tenth edge 210 with the greatest span of length. The eighth edge 208 is the edge of the interior left panel 122 selected from the group consisting of the eighth edge 208, the tenth edge 210, and the twelfth edge 212 with the greatest span of length.

The ninth edge 209 is the edge of the interior left panel 122 that is distal from the seventh edge 207. The twelfth edge 212 is the edge of the interior left panel 122 that is distal from the eighth edge 208. The position of the eleventh edge 211 is between the seventh edge 207 and the ninth edge 209. The tenth edge 210 is between the twelfth edge 212 and the eighth edge 208.

The vertex formed by the seventh edge 207 and the eighth edge 208 forms a right angle. The vertex formed by the eighth edge 208 and the ninth edge 209 forms a right angle. The vertex formed by the ninth edge 209 and the tenth edge 210 forms a right angle. The vertex formed by the tenth edge 210 and the eleventh edge 211 forms a right angle. The vertex formed by the eleventh edge 211 and the twelfth edge 212 forms a right angle. The vertex formed by the twelfth edge 212 and the seventh edge 207 forms a right angle.

The posterior panel 123 is a textile-based sheeting. The posterior panel 123 is formed in a rectilinear shape. The posterior panel 123 is the portion of the garment 102 that encloses the posterior region of the pelvis of the patient 104. The posterior panel 123 comprises a thirteenth edge 213, a fourteenth edge 214, a fifteenth edge 215, a sixteenth edge 216, a seventeenth edge 217, an eighteenth edge 218, a nineteenth edge 219, and a twentieth edge 220.

The thirteenth edge 213, the fifteenth edge 215, the seventeenth edge 217, and the nineteenth edge 219 of the posterior panel 123 are parallel edges. The fourteenth edge 214, the sixteenth edge 216, the eighteenth edge 218, and the nineteenth edge 219 of the posterior panel 123 are parallel edges. The thirteenth edge 213 is the edge of the posterior panel 123 selected from the group consisting of the thirteenth edge 213, the fifteenth edge 215, the seventeenth edge 217, and the nineteenth edge 219 with the greatest span of length.

The span of the length of the fourteenth edge 214 equals the span of the length of the twentieth edge 220. The span of the length of the sixteenth edge 216 equals the span of the length of the eighteenth edge 218. The span of the length of the fifteenth edge 215 equals the span of the length of the nineteenth edge 219. The sixteenth edge 216 is positioned between the eighteenth edge 218 and the fourteenth edge 214. The eighteenth edge 218 is positioned between the sixteenth edge 216 and the twentieth edge 220. The span of the length of the distance between the thirteenth edge 213 and the nineteenth edge 219 equals the span of the length of the distance between the thirteenth edge 213 and the fifteenth edge 215. The span of the length of the distance between the thirteenth edge 213 and the seventeenth edge 217 is less than the span of the length of the distance between the thirteenth edge 213 and the fifteenth edge 215.

The vertex formed by the thirteenth edge 213 and the fourteenth edge 214 forms a right angle. The vertex formed by the fourteenth edge 214 and the fifteenth edge 215 forms a right angle. The vertex formed by the fifteenth edge 215 and the sixteenth edge 216 forms a right angle. The vertex formed by the sixteenth edge 216 and the seventeenth edge 217 forms a right angle. The vertex formed by the seventeenth edge 217 and the eighteenth edge 218 forms a right angle. The vertex formed by the eighteenth edge 218 and the nineteenth edge 219 forms a right angle. The vertex formed by the nineteenth edge 219 and the twentieth edge 220 forms a right angle. The vertex formed by the twentieth edge 220 and the thirteenth edge 213 forms a right angle.

The elastic webbing 124 is a textile-based structure. The elastic webbing 124 secures the garment 102 to the waist of the patient 104. The elastic webbing 124 is defined elsewhere in this disclosure.

The elastic webbing 124 acts as a spring. Specifically, when a force is applied to both ends of the elastic webbing 124 in a direction parallel to the major axis of the elastic webbing 124, the applied force elongates the span of the end to end length the elastic webbing 124 in the direction parallel to the center axis of the elastic webbing 124. The elasticity of the elastic webbing 124 creates a force that opposes the displacement created by the applied force. The elasticity of the elastic webbing 124 returns the elastic webbing 124 to return to its relaxed shape. When the elongated elastic webbing 124 is wrapped around a patient 104, the patient 104 will prevent the elastic webbing 124 from returning to its relaxed shape. In this circumstance, the elastic webbing 124 will apply a force projecting radially away from the major axis of the elastic webbing 124 and through the lateral face of the elastic webbing 124 and against the patient 104 that binds the elastic webbing 124 to the patient 104.

The elastic webbing 124 comprises a first webbing edge 221, a second webbing edge 222, a third webbing edge 223, and a fourth webbing edge 224. The first webbing edge 221 is the end of the disk structure of the elastic webbing 124 with the least span of length. The fourth webbing edge 224 is the end of the disk structure of the elastic webbing 124 with the greatest span of length. The third webbing edge 223 is the end of the disk structure of the elastic webbing 124 that is distal from the first webbing edge 221. The second webbing edge 222 is the end of the disk structure of the elastic webbing 124 that is distal from the fourth webbing edge 224.

The plurality of seams 125 is a seam selected from the group consisting of a sewn seam and an adhesive seams. Each of the plurality of seams 125 forms an attachment selected from the group consisting of: a) attaching the interior right panel 121 to the posterior panel 123; b) attaching the interior left panel 122 to the posterior panel 123; c) attaching the interior right panel 121 to the interior left panel 122; d) attaching the elastic webbing 124 to the interior right panel 121, the interior left panel 122, and the posterior panel 123; e) attaching the elastic webbing 124 to itself; f) attaching a portion of a quick release buckle selected from the plurality of quick release buckles 103 to a panel selected from the group consisting of the interior right panel 121 and the interior left panel 122; g) attaching a portion of a quick release buckle selected from the plurality of quick release buckles 103 to the codpiece 111.

The plurality of seams 125 comprises a first seam 231, a second seam 232, a third seam 233, a fourth seam 234, a fifth seam 235, a sixth seam 236, a seventh seam 237, an eighth seam 238, a ninth seam 239, a tenth seam 240, an eleventh seam 241, a twelfth seam 242, a thirteenth seam 243, a fourteenth seam 244, a fifteenth seam 245, a sixteenth seam 246, and a seventeenth seam 247.

The first seam 231 is a sewn seam. The second seam 232 is a sewn seam. The third seam 233 is a sewn seam. The fourth seam 234 is a sewn seam. The fifth seam 235 is a sewn seam. The sixth seam 236 is a sewn seam. The seventh seam 237 is a sewn seam. The eighth seam 238 is a sewn seam. The ninth seam 239 is a sewn seam. The tenth seam 240 is a sewn seam. The eleventh seam 241 is a sewn seam. The twelfth seam 242 is a sewn seam. The thirteenth seam 243 is a sewn seam. The fourteenth seam 244 is a sewn seam.

The fifteenth seam 245 is an adhesive seam. The sixteenth seam 246 is an adhesive seam. The seventeenth seam 247 is an adhesive seam.

The aperture 126 is an opening that is formed between the second edge 202 of the interior right panel 121 and the twelfth edge 212 of the interior left panel 122. The aperture 126 is bounded by the sixth seam 236, the seventh seam 237, the eighth seam 238, and the ninth seam 239. The aperture 126 is sized such that the receiving structure 141 of the condom catheter 112 of the catheter structure 101 inserts into the garment 102 through the aperture 126.

Each of the plurality of quick release buckles 103 is a quick release buckle. The quick release buckle is defined elsewhere in this disclosure. Each of the plurality of quick release buckles 103 is a fastening structure that removably attaches the catheter structure 101 to the garment 102. The plurality of quick release buckles 103 comprises a first quick release buckle 131, a second quick release buckle 132, and a third quick release buckle 133.

The first quick release buckle 131 is a quick release buckle that attaches the codpiece 111 of the catheter structure 101 to the interior right panel 121 of the garment 102. The first quick release buckle 131 comprises a first male connector 151 and a first female connector 161. The first male connector 151 is the male connector of the first quick release buckle 131. The first male connector 151 attaches to the face of the interior right panel 121 of the garment 102. The first female connector 161 is the female connector of the first quick release buckle 131. The first female connector 161 attaches to the exterior surface of the codpiece 111 of the catheter structure 101.

The second quick release buckle 132 is a quick release buckle that attaches the codpiece 111 of the catheter structure 101 to the interior left panel 122 of the garment 102. The second quick release buckle 132 comprises a second male connector 152 and a second female connector 162. The second male connector 152 is the male connector of the second quick release buckle 132. The second male connector 152 attaches to the face of the interior left panel 122 of the garment 102. The second female connector 162 is the female connector of the second quick release buckle 132. The second female connector 162 attaches to the exterior surface of the codpiece 111 of the catheter structure 101.

The third quick release buckle 133 is a quick release buckle that attaches the codpiece 111 of the catheter structure 101 to the interior left panel 122 of the garment 102. The third quick release buckle 133 comprises a third male connector 153 and a third female connector 163. The third male connector 153 is the male connector of the third quick release buckle 133. The third male connector 153 attaches to the face of the interior left panel 122 of the garment 102. The third female connector 163 is the female connector of the third quick release buckle 133. The third female connector 163 attaches to the exterior surface of the codpiece 111 of the catheter structure 101.

The following four paragraphs describe the assembly of the invention 100.

The first seam 231 attaches the sixth edge 206 of the interior right panel 121 to the twentieth edge 220 of the posterior panel 123. The second seam 232 attaches the fourth edge 204 of the interior right panel 121 to the eighteenth edge 218 of the posterior panel 123. The third seam 233 attaches the third edge 203 of the interior right panel 121 and the eleventh edge 211 of the interior left panel 122 to the seventeenth edge 217 of the posterior panel 123. The fourth seam 234 attaches the tenth edge 210 of the interior left panel 122 to the sixteenth edge 216 of the posterior panel 123. The fifth seam 235 attaches the eighth edge 208 of the interior left panel 122 to the fourteenth edge 214 of the posterior panel 123.

The sixth seam 236 attaches a portion of the twelfth edge 212 of the interior left panel 122 to the face of the interior right panel 121. The seventh seam 237 attaches a portion of the second edge 202 of the interior right panel 121 to the face of the interior left panel 122. The eighth seam 238 attaches a portion of the twelfth edge 212 of the interior left panel 122 to the face of the interior right panel 121. The ninth seam 239 attaches a portion of the second edge 202 of the interior right panel 121 to the face of the interior left panel 122.

The tenth seam 240 simultaneously attaches the fourth webbing edge 224 of the elastic webbing 124 to the first edge 201 of the interior right panel 121, the seventh edge 207 of the interior left panel 122, and the thirteenth edge 213 of the posterior panel 123. The elastic webbing 124 is placed under tension when the tenth seam 240 is formed. The eleventh seam 241 attaches the first webbing edge 221 of the elastic webbing 124 to the third webbing edge 223 of the elastic webbing 124 to form a loop structure.

The twelfth seam 242 attaches the first male connector 151 to the face of the interior right panel 121. The thirteenth seam 243 attaches the second male connector 152 to the face of the interior left panel 122. The fourteenth seam 244 attaches the third male connector 153 to the face of the interior left panel 122. The fifteenth seam 245 attaches the first female connector 161 to the face of the codpiece 111. The sixteenth seam 246 attaches the second female connector 162 to the face of the codpiece 111. The seventeenth seam 247 attaches the third female connector 163 to the face of the codpiece 111.

The following definitions were used in this disclosure:

Adhesive: As used in this disclosure, an adhesive is a chemical substance that can be used to adhere two or more objects to each other. Types of adhesives include, but are not limited to, epoxies, polyurethanes, polyimides, or cyanoacrylates, silicone, or latex based adhesives.

Bind: As used in this disclosure, to bind is a verb that means to tie or secure a first object to a second object using a strap, cord or webbing.

Bladder: As used in this disclosure, a bladder is a fluid impermeable structure. The internal volume of the structure can be varied by: a) varying the pressure and/or quantity of a fluid contained within the bladder; or b) varying the quantity of a liquid contained within the bladder. Bladders are commonly used for the storage of a fluid and as a cushion.

Buckle: As used in this disclosure, a buckle is a fastening device that is used for joining a first loose end of a strap to a second loose end of the same strap or a different strap. A buckle further comprises a male connector that is attached to a first loose end and a female connector that is attached to a second loose end. The male connector has a pin or other structure that is generally caught by a structure formed in the female connector.

Catheter: As used in this disclosure, a catheter is a flexible tube that is inserted into the body through which images may be captured and fluids may be introduced into or removed from the body. Endoscope is a synonym for catheter. A condom catheter is a tubular structure that is designed be worn over the penis of a male patient. The condom catheter transports captured excretions away from the male patient.

Center: As used in this disclosure, a center is a point that is: 1) the point within a circle that is equidistant from all the points of the circumference; 2) the point within a regular polygon that is equidistant from all the vertices of the regular polygon; 3) the point on a line that is equidistant from the ends of the line; 4) the point, pivot, or axis around which something revolves; or, 5) the centroid or first moment of an area or structure. In cases where the appropriate definition or definitions are not obvious, the fifth option should be used in interpreting the specification.

Center Axis: As used in this disclosure, the center axis is the axis of a cylinder or a prism. The center axis of a prism is the line that joins the center point of the first congruent face of the prism to the center point of the second corresponding congruent face of the prism. The center axis of a pyramid refers to a line formed through the apex of the pyramid that is perpendicular to the base of the pyramid. When the center axes of two cylinder, prism or pyramidal structures share the same line they are said to be aligned. When the center axes of two cylinder, prism or pyramidal structures do not share the same line they are said to be offset.

Cord: As used in this disclosure, a cord is a long, thin, flexible, and prism shaped string, line, rope, or wire. Cords are made from yarns, piles, or strands of material that are braided or twisted together or from a monofilament (such as fishing line). Cords have tensile strength but are too flexible to provide compressive strength and are not suitable for use in pushing objects. String, line, cable, and rope are synonyms for cord.

Elastic: As used in this disclosure, an elastic is a material or object that deforms when a force is applied to it and that is able to return to its relaxed shape after the force is removed. A material that exhibits these qualities is also referred to as an elastomeric material. A material that does not exhibit these qualities is referred to as inelastic or an inelastic material.

Elastic Webbing: As used in this disclosure, an elastic webbing is a webbing that contains elastic yarns as some of the yarns that make up the webbing. An elastic webbing is constructed such that the elastic webbing will stretch when a force is applied and will return to its original shape when after the force is removed.

Elimination: As used in this disclosure, an elimination refers to a solid phase discharge from a biological entity.

Excretion: As used in this disclosure, an excretion refers to a liquid phase discharge from a biological entity.

Fitting: As used in this disclosure, a fitting is a component that is attached to a first object. The fitting is used to forming a fluidic connection between the first object and a second object.

Fluid: As used in this disclosure, a fluid refers to a state of matter wherein the matter is capable of flow and takes the shape of a container it is placed within. The term fluid commonly refers to a liquid or a gas.

Fluidic Connection: As used in this disclosure, a fluidic connection refers to a tubular structure that transports a fluid from a first object to a second object. Methods to design and use a fluidic connections are well-known and documented in the mechanical, chemical, and plumbing arts.

Force of Gravity: As used in this disclosure, the force of gravity refers to a vector that indicates the direction of the pull of gravity on an object at or near the surface of the earth.

Hose: As used in this disclosure, a hose is a flexible hollow prism-shaped device that is used for transporting liquids and gases. When referring to a hose in this disclosure, the terms inner dimension and outer dimension are used as they would be used by those skilled in the plumbing arts.

Inferior: As used in this disclosure, the term inferior refers to a directional reference that is parallel to and in the same direction as the force of gravity when an object is positioned or used normally.

Interior: As used in this disclosure, interior is a term that is used to refer to the front side or direction of a structure. When comparing two objects, the interior object is the object that is closer to the front of the structure.

Liquid: As used in this disclosure, a liquid refers to a state (phase) of matter that is fluid and that maintains, for a given pressure, a fixed volume that is independent of the volume of the container.

Major and Minor Axes: As used in this disclosure, the major and minor axes refer to a pair of perpendicular axes that are defined within a structure. The length of the major axis is always greater than or equal to the length of the minor axis. The major axis is always the longest diameter of the structure. The major and minor axes intersect at the center of the structure. The major axis is always parallel to the longest edge of a rectangular structure.

Patient: As used in this disclosure, a patient is a person who is designated to receive a medical treatment, therapy or service. The term patient may be extended to an animal when used within the context of the animal receiving veterinary treatment or services.

Pelvis: As used in this disclosure, the pelvis refers to a bone structure near the base of the spine to which buttocks and the legs are joined. As used in this disclosure, the term pelvis is a more generally expanded to describe the above described region of the body. As used in this disclosure, the adjectival form of pelvis is pelvic.

Posterior: As used in this disclosure, posterior is a term that is used to refer to the side of an object that is distal or in the opposite direction of the interior side. When comparing two items, the posterior item is the item that is distal from the interior of the object.

Prism: As used in this disclosure, a prism is a three-dimensional geometric structure wherein: 1) the form factor of two faces of the prism are congruent; and, 2) the two congruent faces are parallel to each other. The two congruent faces are also commonly referred to as the ends of the prism. The surfaces that connect the two congruent faces are called the lateral faces. In this disclosure, when further description is required a prism will be named for the geometric or descriptive name of the form factor of the two congruent faces. If the form factor of the two corresponding faces has no clearly established or well-known geometric or descriptive name, the term irregular prism will be used. The center axis of a prism is defined as a line that joins the center point of the first congruent face of the prism to the center point of the second corresponding congruent face of the prism. The center axis of a prism is otherwise analogous to the center axis of a cylinder. A prism wherein the ends are circles is commonly referred to as a cylinder.

Quick Release Buckle: As used in this disclosure, a quick release buckle is a specific type of buckle wherein the buckle can be readily and easily disconnected by pressing a button or pinching one of the ends of the quick release buckle. Quick release buckles are readily and commercially available.

Rectilinear: As used in this disclosure, rectilinear is an adjective that is used to describe an object that: 1) moves in a straight line or lines; 2) consists of a straight line or lines; 3) is bounded by a straight line or lines; or, 4) is otherwise characterized by a straight line or lines.

Relaxed Shape: As used in this disclosure, a structure is considered to be in its relaxed state when no shear, strain, or torsional forces are being applied to the structure.

Seam: As used in this disclosure, a seam is a joining of: 1) a first textile to a second textile; 2) a first sheeting to a second sheeting; or, 3) a first textile to a first sheeting. Potential methods to form seams include, but are not limited to, a sewn seam, a heat bonded seam, an ultrasonically bonded seam, a laser seam, or a seam formed using an adhesive.

Sewn Seam: As used in this disclosure, a sewn seam a method of attaching two or more layers of textile, leather, or other material through the use of a thread, a yarn, or a cord that is repeatedly inserted and looped through the two or more layers of textile, leather, or other material.

Sheeting: As used in this disclosure, a sheeting is a material, such as a paper, textile, a plastic, or a metal foil, in the form of a thin flexible layer or layers. The sheeting forms a disk structure. The two surfaces of the sheeting with the greatest surface area are called the faces of the sheeting.

Superior: As used in this disclosure, the term superior refers to a directional reference that is parallel to and in the opposite direction of the force of gravity when an object is positioned or used normally.

Textile: As used in this disclosure, a textile is a material that is woven, knitted, braided or felted. Synonyms in common usage for this definition include fabric and cloth. The two surfaces of the textile with the greatest surface area are called the faces of the textile.

Tube: As used in this disclosure, a tube is a hollow prism-shaped device formed with two open ends. The tube is used for transporting liquids and gases. The line that connects the center of the first congruent face of the prism to the center of the second congruent face of the prism is referred to as the center axis of the tube or the centerline of the tube. When two tubes share the same centerline they are said to be aligned. When the centerlines of two tubes are perpendicular to each other, the tubes are said to be perpendicular to each other. In this disclosure, the terms inner dimensions of a tube and outer dimensions of a tube are used as they would be used by those skilled in the plumbing arts.

Underclothing: As used in this disclosure, underclothing refers to garments that are intended to be worn next to the skin. Underclothing is often worn in conjunction with an outer layer of clothing.

Urethra: As used in this disclosure, the urethra is a biological structure found in mammals. The urethra is a tubular structure that transports urine from the bladder for discharge into the environment.

Webbing: As used in this disclosure, a webbing is a strong, close woven or knitted fabric that is used for straps or belting. As used in this disclosure, webbing is a fully formed material that is only cut to length for use. Webbing is not formed by cutting broader materials into strips. Webbings have tensile strength but are too flexible to provide compressive strength and are not suitable for use in pushing objects. The shape of a webbing is approximated by a rectangular disk shape. The two surfaces of a webbing with the greatest surface area are called the faces of the webbing.

With respect to the above description, it is to be realized that the optimum dimensional relationship for the various components of the invention described above and in FIGS. 1 through 6 include variations in size, materials, shape, form, function, and manner of operation, assembly and use, are deemed readily apparent and obvious to one skilled in the art, and all equivalent relationships to those illustrated in the drawings and described in the specification are intended to be encompassed by the invention.

It shall be noted that those skilled in the art will readily recognize numerous adaptations and modifications which can be made to the various embodiments of the present invention which will result in an improved invention, yet all of which will fall within the spirit and scope of the present invention as defined in the following claims. Accordingly, the invention is to be limited only by the scope of the following claims and their equivalents.

The inventor claims:

1. A medical device comprising
a catheter structure, a garment, and a plurality of quick release buckles;
wherein the plurality of quick release buckles attaches the catheter structure to the garment;
wherein the medical device is an apparel item that is
worn as underclothing;
wherein the catheter structure is a medical collection device that is configured to attach and be worn over a pelvis of a male patient in order to capture and store urine eliminated by said patient;
wherein the catheter structure comprises a codpiece and a condom catheter;
wherein the codpiece is a bladder;
wherein the condom catheter is a medical collection device;
wherein the condom catheter is adapted to form a fluidic connection between a urethra of the patient and the codpiece;
wherein the plurality of quick release buckles attaches the codpiece to the garment;
wherein the condom catheter transports the received excretions to the codpiece;
wherein the condom catheter comprises a receiving structure, a tube, and a fitting;
wherein the receiving structure is a tubular structure;
wherein the receiving structure is configured to receive the excretions released from the urethra and transports the excretions to the tube;
wherein the tube is a hose;
wherein the tube forms a fluidic connection between the receiving structure and the fitting;
wherein the tube transports the received excretions to the fitting;
wherein the fitting is a mechanical structure that attaches the tube to the codpiece;
wherein the fitting forms a fluidic connection between the tube and the interior of the bladder structure of the codpiece such that the excretions transported by the tube are released into the codpiece.

2. The medical device according to claim 1
wherein the garment is an item of underclothing;
wherein the garment is a textile-based structure;
wherein the garment is configured to secure the catheter structure to the patient.

3. The medical device according to claim 2
wherein each of the plurality of quick release buckles is a quick release buckle;
wherein each of the plurality of quick release buckles is a fastening structure that removably attaches the catheter structure to the garment.

4. (canceled)

5. The medical device according to claim 3
wherein the garment comprises an interior right panel, an interior left panel, a posterior panel, an elastic webbing, and a plurality of seams;
wherein the interior right panel is the portion of the garment that is configured to enclose the right interior region of the pelvis of the patient;
wherein the interior left panel is the portion of the garment that is configured to enclose the left interior region of the pelvis of the patient;
wherein the posterior panel is the portion of the garment that is configured to enclose the posterior region of the pelvis of the patient;
wherein the elastic webbing is configured to secure the garment to the waist of the patient;
wherein each of plurality of seams forms an attachment selected from the group consisting of: a) attaching the interior right panel to the posterior panel; b) attaching the interior left panel to the posterior panel; c) attaching the interior right panel to the interior left panel; d) attaching the elastic webbing to the interior right panel, the interior left panel, and the posterior panel; e) attaching the elastic webbing to itself; f) attaching a portion of a quick release buckle selected from the plurality of quick release buckles to a panel selected from the group consisting of the interior right panel and the interior left panel; g) attaching a portion of a quick release buckle selected from the plurality of quick release buckles to the codpiece.

6. The medical device according to claim 5
wherein the plurality of quick release buckles comprises a first quick release buckle, a second quick release buckle, and a third quick release buckle;
wherein the first quick release buckle is a quick release buckle that attaches the codpiece of the catheter structure to the interior right panel of the garment;
wherein the second quick release buckle is a quick release buckle that attaches the codpiece of the catheter structure to the interior left panel of the garment;
wherein the third quick release buckle is a quick release buckle that attaches the codpiece of the catheter structure to the interior left panel of the garment.

7. (canceled)

8. The medical device according to claim 6
wherein the interior right panel is a textile-based sheeting;
wherein the interior right panel is formed in a rectilinear shape;
wherein the interior left panel is a textile-based sheeting;
wherein the interior left panel is formed in a rectilinear shape;
wherein the posterior panel is a textile-based sheeting;
wherein the posterior panel is formed in a rectilinear shape;
wherein the elastic webbing is a textile-based structure;
wherein the elastic webbing acts as a spring;
wherein the elastic webbing binds itself to the patient;
wherein the plurality of seams is a seam selected from the group consisting of a sewn seam and an adhesive seams.

9. The medical device according to claim 8
wherein the interior right panel comprises a first edge, a second edge, a third edge, a fourth edge, a fifth edge, and a sixth edge;
wherein the first edge, the third edge, and the fifth edge of the interior right panel are parallel edges;
wherein the second edge, the fourth edge, and the sixth edge of the interior right panel are parallel edges;
wherein the first edge is the edge of the interior right panel selected from the group consisting of the first edge, the third edge, and the fourth edge with the greatest span of length;
wherein the sixth edge is the edge of the interior right panel selected from the group consisting of the second edge, the fourth edge, and the sixth edge with the greatest span of length;
wherein the fifth edge is the edge of the interior right panel that is distal from the first edge;
wherein the second edge is the edge of the interior right panel that is distal from the sixth edge;
wherein the position of the third edge is between the first edge and the fifth edge;
wherein the fourth edge is between the sixth edge and the second edge;
wherein the vertex formed by the first edge and the second edge forms a right angle;
wherein the vertex formed by the second edge and the third edge forms a right angle;
wherein the vertex formed by the third edge and the fourth edge forms a right angle;
wherein the vertex formed by the fourth edge and the fifth edge forms a right angle;
wherein the vertex formed by the fifth edge and the sixth edge forms a right angle;
wherein the vertex formed by the sixth edge and the first edge forms a right angle.

10. The medical device according to claim 9
wherein the interior left panel comprises a seventh edge, an eighth edge, a ninth edge, a tenth edge, an eleventh edge, and a twelfth edge;
wherein the seventh edge, the ninth edge, and the eleventh edge of the interior left panel are parallel edges;
wherein the eighth edge, the tenth edge, and the twelfth edge of the interior left panel are parallel edges;
wherein the seventh edge is the edge of the interior left panel selected from the group consisting of the seventh edge, the ninth edge, and the tenth edge with the greatest span of length;
wherein the eighth edge is the edge of the interior left panel selected from the group consisting of the eighth edge, the tenth edge, and the twelfth edge with the greatest span of length;
wherein the ninth edge is the edge of the interior left panel that is distal from the seventh edge;
wherein the twelfth edge is the edge of the interior left panel that is distal from the eighth edge;
wherein the position of the eleventh edge is between the seventh edge and the ninth edge;
wherein the tenth edge is between the twelfth edge and the eighth edge;
wherein the vertex formed by the seventh edge and the eighth edge forms a right angle;
wherein the vertex formed by the eighth edge and the ninth edge forms a right angle;
wherein the vertex formed by the ninth edge and the tenth edge forms a right angle;
wherein the vertex formed by the tenth edge and the eleventh edge forms a right angle;
wherein the vertex formed by the eleventh edge and the twelfth edge forms a right angle;
wherein the vertex formed by the twelfth edge and the seventh edge forms a right angle.

11. The medical device according to claim 10
wherein the posterior panel comprises a thirteenth edge, a fourteenth edge, a fifteenth edge, a sixteenth edge, a seventeenth edge, an eighteenth edge, a nineteenth edge, and a twentieth edge;
wherein the thirteenth edge, the fifteenth edge, the seventeenth edge, and the nineteenth edge of the posterior panel are parallel edges;
wherein the fourteenth edge, the sixteenth edge, the eighteenth edge, and the nineteenth edge of the posterior panel are parallel edges;
wherein the thirteenth edge is the edge of the posterior panel selected from the group consisting of the thirteenth edge, the fifteenth edge, the seventeenth edge, and the nineteenth edge with the greatest span of length;
wherein the span of the length of the fourteenth edge equals the span of the length of the twentieth edge;
wherein the span of the length of the sixteenth edge equals the span of the length of the eighteenth edge;
wherein the span of the length of the fifteenth edge equals the span of the length of the nineteenth edge;
wherein the sixteenth edge is positioned between the eighteenth edge and the fourteenth edge;
wherein the eighteenth edge is positioned between the sixteenth edge and the twentieth edge;
wherein the span of the length of the distance between the thirteenth edge and the nineteenth edge equals the span of the length of the distance between the thirteenth edge and the fifteenth edge;
wherein the span of the length of the distance between the thirteenth edge and the seventeenth edge is less than the span of the length of the distance between the thirteenth edge and the fifteenth edge;
wherein the vertex formed by the thirteenth edge and the fourteenth edge forms a right angle;
wherein the vertex formed by the fourteenth edge and the fifteenth edge forms a right angle;
wherein the vertex formed by the fifteenth edge and the sixteenth edge forms a right angle;
wherein the vertex formed by the sixteenth edge and the seventeenth edge forms a right angle;
wherein the vertex formed by the seventeenth edge and the eighteenth edge forms a right angle;
wherein the vertex formed by the eighteenth edge and the nineteenth edge forms a right angle;
wherein the vertex formed by the nineteenth edge and the twentieth edge forms a right angle;
wherein the vertex formed by the twentieth edge and the thirteenth edge forms a right angle.

12. The medical device according to claim 11
wherein the elastic webbing comprises a first webbing edge, a second webbing edge, a third webbing edge, and a fourth webbing edge;
wherein the first webbing edge is the end of a disk structure of the elastic webbing with the least span of length;
wherein the fourth webbing edge is the end of a disk structure of the elastic webbing with the greatest span of length;
wherein the third webbing edge is the end of a disk structure of the elastic webbing that is distal from the first webbing edge;
wherein the second webbing edge is the end of a disk structure of the elastic webbing that is distal from the fourth webbing edge.

13. The medical device according to claim 12
wherein the plurality of seams comprises a first seam, a second seam, a third seam, a fourth seam, a fifth seam, a sixth seam, a seventh seam, an eighth seam, a ninth seam, a tenth seam, an eleventh seam, a twelfth seam, a thirteenth seam, a fourteenth seam, a fifteenth seam, a sixteenth seam, and a seventeenth seam;
wherein the first seam is a sewn seam;
wherein the second seam is a sewn seam;
wherein the third seam is a sewn seam;
wherein the fourth seam is a sewn seam;
wherein the fifth seam is a sewn seam;
wherein the sixth seam is a sewn seam;
wherein the seventh seam is a sewn seam;
wherein the eighth seam is a sewn seam;
wherein the ninth seam is a sewn seam;
wherein the tenth seam is a sewn seam;
wherein the eleventh seam is a sewn seam;
wherein the twelfth seam is a sewn seam;
wherein the thirteenth seam is a sewn seam;
wherein the fourteenth seam is a sewn seam;
wherein the fifteenth seam is an adhesive seam;
wherein the sixteenth seam is an adhesive seam;
wherein the seventeenth seam is an adhesive seam.

14. The medical device according to claim 13
wherein the first quick release buckle comprises a first male connector and a first female connector;
wherein the first male connector is the male connector of the first quick release buckle;
wherein the first male connector attaches to the face of the interior right panel of the garment;
wherein the first female connector is the female connector of the first quick release buckle;
wherein the first female connector attaches to the exterior surface of the codpiece of the catheter structure.

15. The medical device according to claim 14
wherein the second quick release buckle comprises a second male connector and a second female connector;
wherein the second male connector is the male connector of the second quick release buckle;
wherein the second male connector attaches to the face of the interior left panel of the garment;
wherein the second female connector is the female connector of the second quick release buckle;
wherein the second female connector attaches to the exterior surface of the codpiece of the catheter structure.

16. The medical device according to claim 15
wherein the third quick release buckle comprises a third male connector and a third female connector;
wherein the third male connector is the male connector of the third quick release buckle;
wherein the third male connector attaches to the face of the interior left panel of the garment;
wherein the third female connector is the female connector of the third quick release buckle;
wherein the third female connector attaches to the exterior surface of the codpiece of the catheter structure.

17. The medical device according to claim 16
wherein the first seam attaches the sixth edge of the interior right panel to the twentieth edge of the posterior panel;
wherein the second seam attaches the fourth edge of the interior right panel to the eighteenth edge of the posterior panel;
wherein the third seam attaches the third edge of the interior right panel and the eleventh edge of the interior left panel to the seventeenth edge of the posterior panel;
wherein the fourth seam attaches the tenth edge of the interior left panel to the sixteenth edge of the posterior panel;
wherein the fifth seam attaches the eighth edge of the interior left panel to the fourteenth edge of the posterior panel;
wherein the sixth seam attaches a portion of the twelfth edge of the interior left panel to the face of the interior right panel;
wherein the seventh seam attaches a portion of the second edge of the interior right panel to the face of the interior left panel;
wherein the eighth seam attaches a portion of the twelfth edge of the interior left panel to the face of the interior right panel;
wherein the ninth seam attaches a portion of the second edge of the interior right panel to the face of the interior left panel.

18. The medical device according to claim 17
wherein the tenth seam simultaneously attaches the fourth webbing edge of the elastic webbing to the first edge of the interior right panel, the seventh edge of the interior left panel, and the thirteenth edge of the posterior panel;
wherein the eleventh seam attaches the first webbing edge of the elastic webbing to the third webbing edge of the elastic webbing to form a loop structure.

19. The medical device according to claim 18
wherein the twelfth seam attaches the first male connector to the face of the interior right panel;
wherein the thirteenth seam attaches the second male connector to the face of the interior left panel;
wherein the fourteenth seam attaches the third male connector to the face of the interior left panel;
wherein the fifteenth seam attaches the first female connector to the codpiece;
wherein the sixteenth seam attaches the second female connector to the codpiece;
wherein the seventeenth seam attaches the third female connector to the codpiece.
